(19) United States
(12) Patent Application Publication
(10) Pub. No.: US 2011/0160712 A1
(43) Pub. Date: Jun. 30, 2011

(54) Laser treatment system and method for producing thermal cavities and energy droplets

(76) Inventors: Nikolai Tankovich, San Diego, CA (US); Alexei Lukashev, San Diego, CA (US)

(21) Appl. No.: 12/590,075

(22) Filed: Nov. 2, 2009

Related U.S. Application Data
(63) Continuation-in-part of application No. 10/890,076, filed on Jul. 12, 2004, now abandoned.

(51) Int. Cl. A61B 18/20

(52) U.S. Cl. 606/9

(57) ABSTRACT

A laser process for tissue treatment. Preferred embodiments include features for producing a first laser beam and a second laser beam and a skin cooler for cooling the surface of a region of skin. The system is designed to utilize the first laser beam for heating a volume of skin tissue below the cooled surface region to a temperature below skin tissue damage threshold. This volume of skin tissue is called a “thermal cavity”. The second laser beam is divided into a plurality of separate laser beams that are directed through separate optical fibers and via separate paths through skin tissue to a single tiny volume of skin tissue within the thermal cavity to heat that tiny volume to a temperature above the damage threshold. This tiny volume is called an energy droplet. Thus tiny regions of tissue are damaged while minimizing or preventing any significant damage to adjacent tissue.

### CROSS REFERENCE TO RELATED APPLICATIONS

This application is a continuation-in-part of U.S. patent application Ser. No. 10/890,076 filed Jul. 12, 2004. That application especially the background section and the description of the first preferred embodiment and related drawings are incorporated herein by reference.

### FIELD OF THE INVENTION

This invention relates to fiber laser systems and in particular to fiber lasers for skin treatment.

### BACKGROUND OF THE INVENTION

### Fiber Lasers

Fiber lasers are lasers made using optical fibers. Light emitting atoms are doped into the core of an optical fiber that confines the light the atoms emit. Optical fibers with mirrors or other reflectors on each end can serve as oscillators. Optical fiber amplifiers are widely used and are similar to the fiber lasers but in the amplifiers there is no oscillation.

### Wavelength Selection

Some wavelengths are very preferentially absorbed in a particular type of tissue when the tissue contains a particular chromophore that has a peak or relatively high absorption at the particular wavelength. Use of a laser beam matched to a peak or relatively high absorption in tissue to treat the tissue is referred to as “selective thermolysis”. Some wavelengths are absorbed relatively uniformly in tissue and when these wavelengths are used to treat the tissue it is referred to as “non-selective thermolysis” or “homogeneous thermolysis”. Choices of wavelengths are important when these lasers are used in medicine and for surgery, tattoo removal, skin peeling and hair removal.

### Tissue Damage

In some medical laser applications, living tissue is intentionally damaged with the laser energy. Damage is typically the result of temperature increases in the skin tissue caused by the energy of the laser beam being absorbed in the skin tissue, the blood vessels, and the blood in the vessels. In some cases tissue adjacent the target tissue can also be damaged. There exist, for normal skin tissue, a skin tissue damage temperature threshold. Temperatures below the threshold produce no significant damage. The threshold depends on time and temperature. For periods of time (for example less than one second) the damage to blood and blood vessels the damage threshold is about 44° C. For other skin tissue the threshold is somewhat higher in the range of about 66 to 72° C.

### The Need

What is needed in a low cost laser system including components to deliver laser energy precisely into small regions of target tissue while minimizing damage to adjacent tissue.

### SUMMARY OF THE INVENTION

The present invention provides a laser process for tissue treatment. Preferred embodiments include the use of a first laser beam and a second laser beam and a skin cooler for cooling the surface of a region of tissue. In preferred embodiments for skin treatments are based on the concept that by intentionally causing damage to a tiny volume of skin below the skin surface of the skin natural skin renewal processes will be initiated in the skin that will extend beyond the region damaged to produce rejuvenated skin.

In preferred embodiments the process includes the steps of illuminating a first region of skin for a first time period with a preheat laser beam with sufficient energy to heat the first region to a temperature-time profile that is close to but below a temperature-time profile that would cause tissue damage. This first region is referred to as a “thermal cavity”. A tiny sub-region of skin located below the skin surface and within the first region of skin is illuminated for a second time period with one or more energy droplet laser beams adapted to apply an additional quantity of heat to the sub-region of skin to increase its temperature-time profile to a temperature-time profile in excess of a temperature-time profile sufficient to cause tissue damage within the sub-region. The additional quantity of heat in the sub-region is referred to as an “energy droplet”. As a result tissue damage in the sub-region stimulates the healing process that subsequently extends beyond the region damaged to produce rejuvenated skin tissue.

Thus tiny regions of tissue are damaged while minimizing or preventing any significant damage to adjacent tissue. Preferred embodiments include a laser hand piece designed to deliver skin surface cooling and the laser beam from the first laser to create the thermal cavity and the laser beams form the second laser a target volume to create the energy droplets. In preferred embodiments the surface cooling is provided with a flow of cold air and the number of laser beams from the second laser are six laser beams. The cooling air in preferred embodiments is at about 0 to 3° C. and may be provided with a commercial off-the-shelf cooling air unit or with simpler unit consisting of a blower unit, an accumulator and a tube coiled in an ice water bath. Other techniques for cooling the skin surface can be used. A simple technique that can be used to pre-cool the skin surface is a metal container containing ice which is pressed against the skin surface prior to laser illumination. The two laser beams can be provided by a single laser system as described in the parent patent application or the two beams may be provided by two separate off-the-shelf laser units.

### Thermal Cavity

In the preferred embodiment the thermal cavity is produced by the cooling air and a single laser beam from a YAP:Nd laser adapted to produce 20 J/cm2, 1079 nm, 1 milli-second pulses. Optical components are provided to produce a beam diameter on a skin surface of about 4 millimeters. The combination of the cooling air and the YAP:Nd laser beam produces a thermal cavity at a relatively uniform temperature of about 40° C. in a region of the skin about 4 mm in diameter and 2 mm to 4 mm below the skin surface. The temperature at the surface is less than 36 C (which is normal body temperature).

### Energy Droplets

The energy droplets are produced by a single erbium-doped fiber laser system described in detail in the parent patent application. This laser system is a master oscillator power amplifier fiber laser system, referred to as a MOPA fiber laser system. In preferred described in detail herein, the laser system operates at a wavelength of 1560 nm with average power of 1.8 watts and pulse durations of 2 microseconds. The output of this second laser system is divided into six separate optical fibers with each fiber carrying microsecond pulses each with a power of about 300 mW. All of the six beams are directed through optical fibers, lenses and prisms to a single small approximately spherical 0.2 mm diameter damage region of the skin within the thermal cavity centered about 2 mm below the skin surface. The temperature in this region is increased to about 47° to 50° C. (about 118° F.) for a few microseconds which is sufficient to produce significant tissue damage in the region. Tissue separated by more than more than 0.06 millimeter from the 0.2 millimeter damage zone is heated to no more than 44° C. (111.2° F.) for a few microseconds. The temperature then quickly drops to about 36° As a result there is no significant tissue damage at distances greater than 0.06 millimeter from the 0.2 millimeter damage region.

### BRIEF DESCRIPTION OF THE DRAWINGS

FIG. 1 is a drawing of a preferred embodiment of the present invention.

FIGS. 1A and 1B show features of laser hand piece of the preferred embodiment.

FIGS. 2A through 2E show approximate temperature contours produced by the laser beams and the cooling air.

FIG. 3 shows the hand piece being applied to a patient.

FIG. 4 is a drawing from the parent application of a laser system useful for producing two separate laser beams.

FIGS. 5A through 5I show alternative techniques for creating energy droplets below the skin surface within an thermal cavity.

### DETAILED DESCRIPTION OF PREFERRED EMBODIMENTS

### Preferred Embodiments

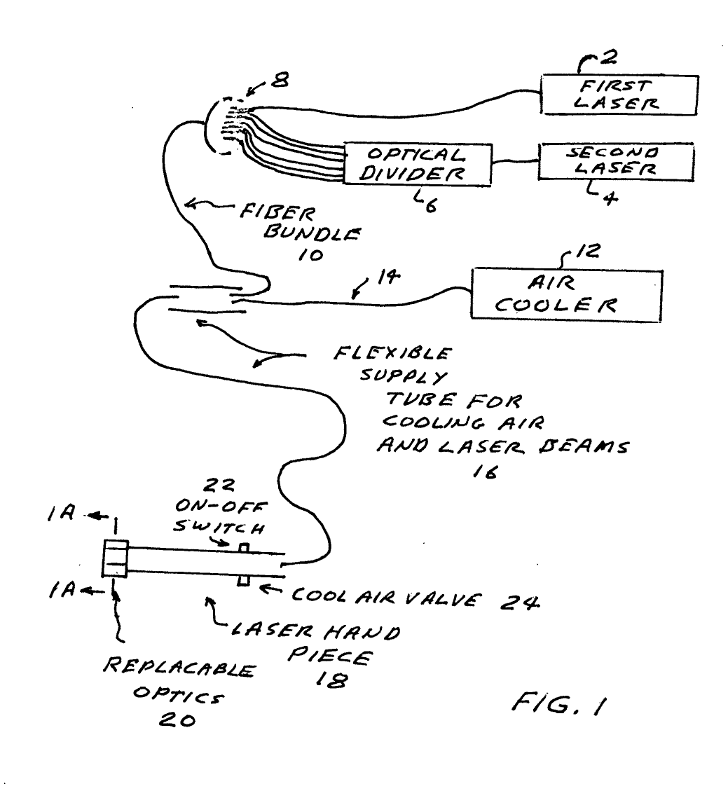

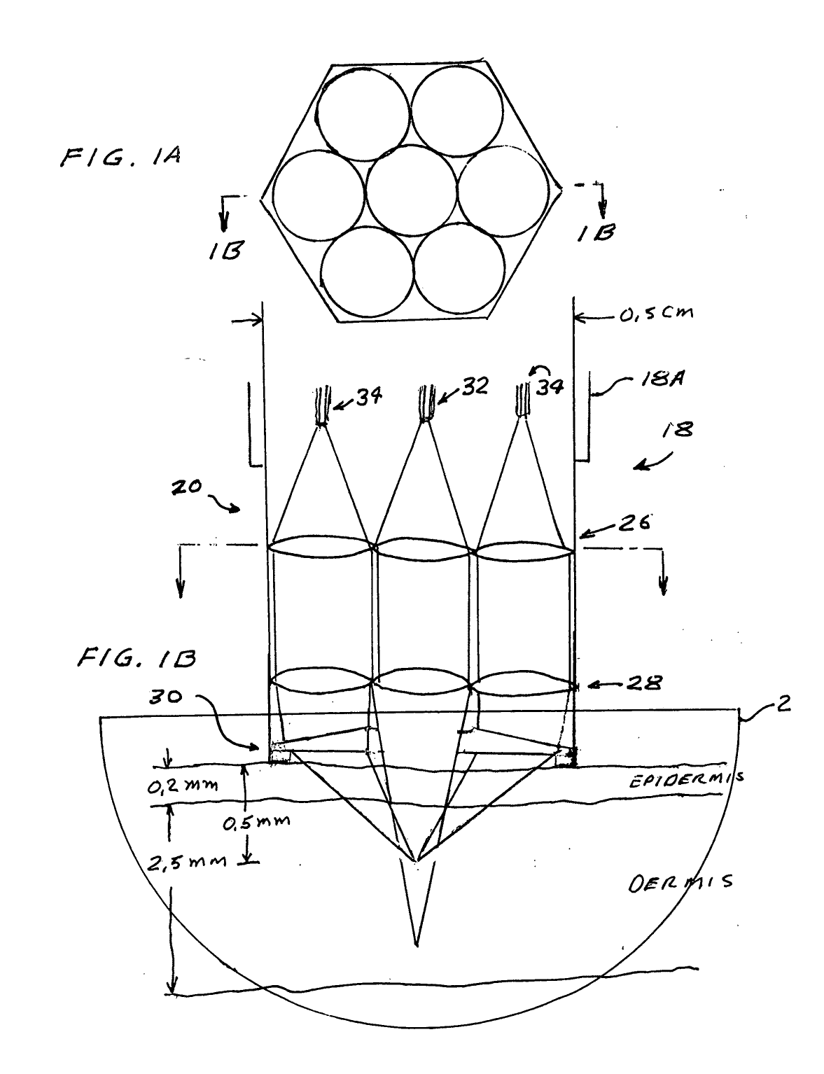

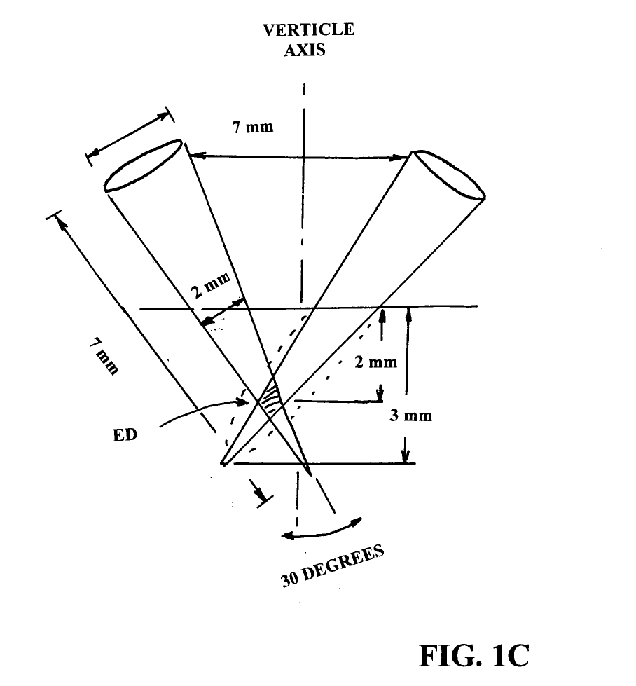

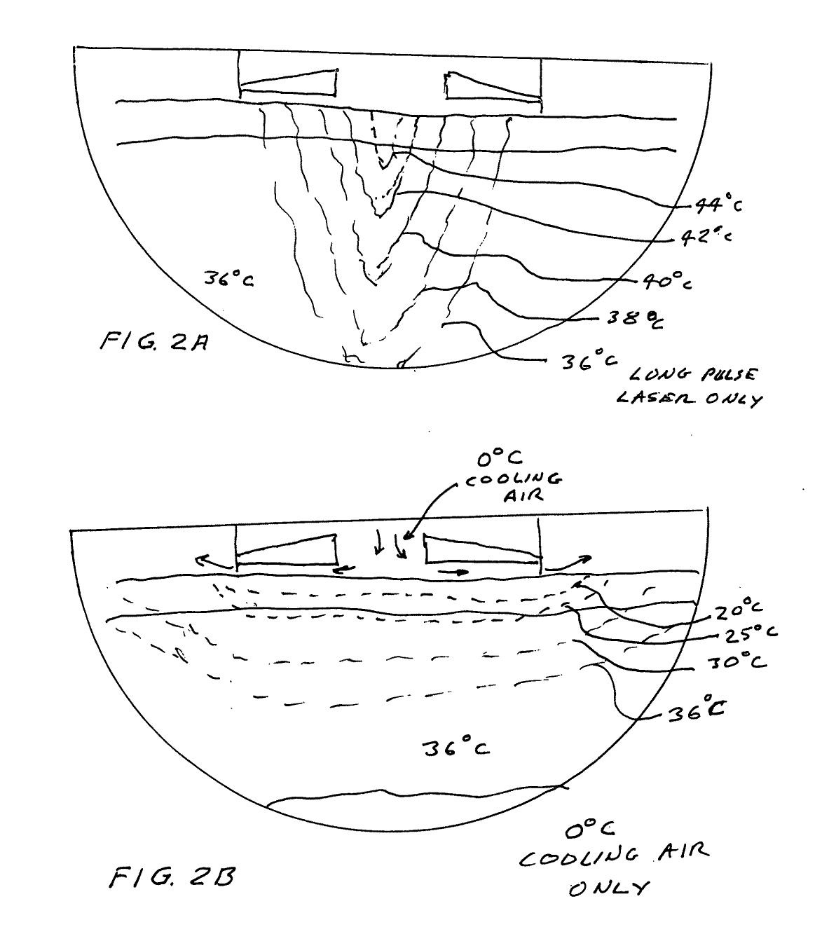

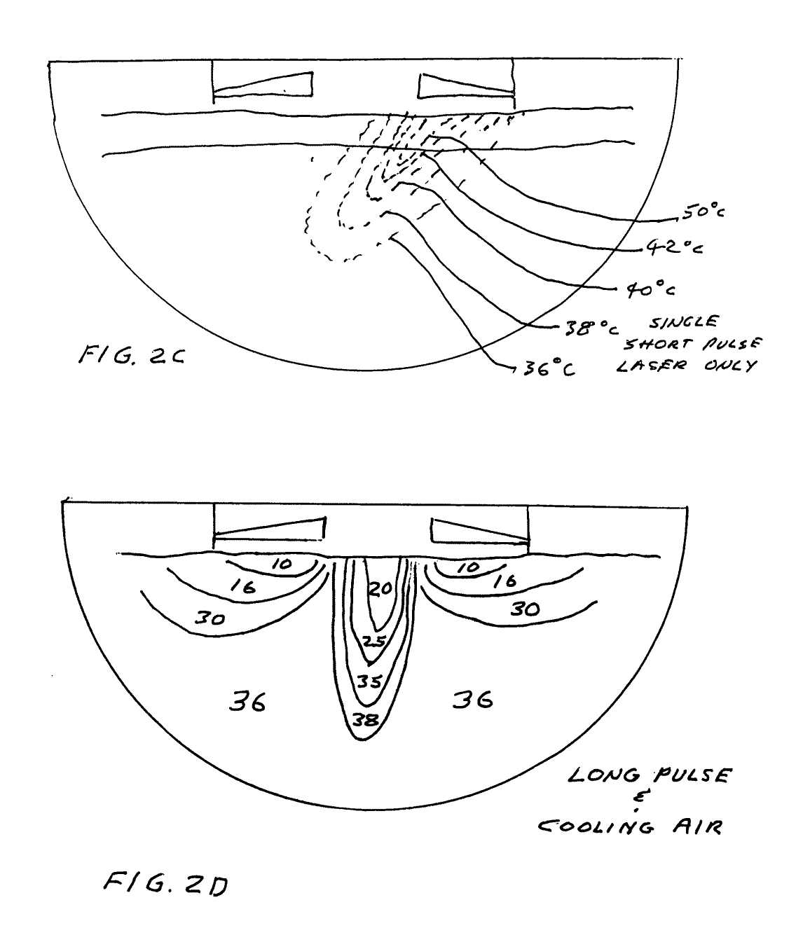

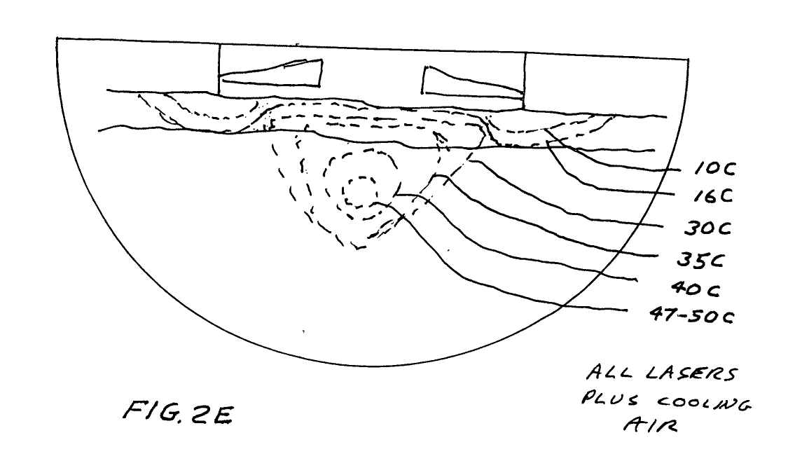

FIGS. 1 through 3 show features of preferred embodiments of the present invention. As shown in FIG. 2 the embodiment includes first laser source 2, second laser source 4, an optical divider 6 for dividing the output of the second laser source into six laser beams each of the six beams are directed into separate optical fibers which fibers are combined in combiner 8 with a single fiber carrying the output of the first laser source 2. The seven fibers are transported in a fiber bundle 10 which is combined cooling air tube 14 carrying approximately zero degrees centigrade cooling air which is cooled air cooler 12. Flexible supply tube 16 carries the seven laser beams and the cooling air to laser hand piece 18 which includes a cooling air valve 24 and a laser on-off switch 22. The laser hand piece 18 includes a replaceable optics unit 20. Details of replaceable optics unit are shown in FIGS. 1A and 1B.

The replaceable optics unit 20 fits in the body 18A if laser hand piece 18 as shown in FIG. 1B. The optics unit includes two lens arrays of seven lenses each for focusing each of the laser beams at desired locations below the skin surface. A prism array of six prisms 28 directs each of the six beams transported in fibers 34 from the second laser to a single location about 0.5 mm below the surface of the skin where each of the beams are focused with the optical combinations of the two lenses and the prism controlling each of the six beams. The beam transported in fiber 32 from the first laser is controlled only by two lenses and is focused at a focal point about 1.0 mm below the skin surface. The reader should note that the beam traces shown in FIG. 1B are approximate traces assuming that the beam were transported in air. Since the beams are instead passing through skin tissue the actual shapes of the beams are substantially different. The general directions of the beams are correctly represented in FIG. 1B. However, the beams will be scattered in the tissue and therefore spread substantially as compared to the traces in FIG. 1B. Also the beams will undergo substantial absorption on the way to their focal location. Applicants have conducted computer modeling to determine the approximate actual shapes of the beams in the skin tissue.

As the photons in the beams are scattered and absorbed in the skin tissue the beam energy is converted into heat increasing the temperature of the skin tissue. The cooling air from air cooler 12 and tube 14 passes out of the center of replaceable optic unit 20 and flows along the surface of the skin as indicated in FIG. 2B and in the process cools the surface of the skin directly below optics unit 20. Based on Applicants' computer analysis Applicants have estimated temperature contour lines separately resulting from each of the seven laser beams and the cooling air. These contour lines are shown in FIG. 2A for the laser beam from the first laser; FIG. 2B for the cooling air; FIG. 2C for each of the six laser beams from the second laser. FIG. 2D shows the contour lines for the combination of the beam from the first laser and the cooling air. This drawing defines the “thermal cavity”. FIG. 2E shows the contour lines for all seven lasers and the cooling air. The 47-50° C. circle in FIG. 2E defines the “energy droplet” produced by this preferred embodiment.

In the preferred embodiment the energy droplet is formed by six crossing beams within the thermal cavity. The wavelength is 1079 nm of the YAP:Nd (Perovskite) laser. Using a lens with f=7 mm and diameter of 4 mm each individual beam is focused with the beam tilted to the vertical axis at 30 degrees. The crossing point of those six beams is about 1 mm above the focus point. The hand piece with those six beams is pre-aligned in the air. For application on skin the hand piece is placed at a distance about 3 mm from the surface of the skin. In this case the crossing point is about 2 mm beneath the surface of the skin. The volume of the energy droplet is about 2 cubic millimeters due to scattering in the skin. In this configuration the laser beam spot size on skin is about 2 mm. The energy delivered to the energy droplet by each of the individual beams is about 112 mJ, so the total energy delivered by the six beams is about 0.672 J. This amount of heat energy from the six beams is sufficient to increase temperature in the 2 cubic millimeter energy droplet volume (corresponding to 1.8 milligrams of skin tissue) enough to produce tissue damage in the volume. Applicants estimate that the temperature rise from all 6 beams is about 9.6° C. For these estimates Applicants used ρ=1 g/cm³ and c=4.2 J/g degrees-° C.

### Heat Dissipation In Thermal Cavity and Energy Droplets

The amount and the rate of energy absorption in light-absorbing chromophores depends on the laser pulse energy and duration of the pulse. The shorter the pulse duration, the higher the temperature rise in the light-absorbing medium if the pulse duration is short enough then at the end of the pulse the absorbed energy is confined well inside the light absorbing chromophore. Alternatively, much of the heat may be dissipated into the surrounding medium if the pulse duration is very long. As an approximation, the energy dissipation distance in a given period of time can be written as:

x=\sqrt{4χτ} (1)

where x in cm—is the energy dissipation distance; τ (in seconds) is the duration of energy dissipation; χ (in cm²/sec) is the thermal diffusivity, which is determined by the mathematic expression χ=K/pc (in which K is in J cm⁻¹s⁻¹° C.⁻¹) is an energy absorption coefficient; ρ (in g/cm³) is the density of the tissue and c (in Jg⁻¹° C.⁻¹) represent the specific heat. Table 1 lists the energy dissipation distances in skin during a period of 100 ns (Q-switched Yb⁺³ and Nd doped fiber laser pulse duration), 100 μs (typical duration of non-Q-switched or free running Nd:YAG/YAP lasers), and 100 ms for long pulse YAP:Nd laser.

TABLE 1
The energy dissipation distance x (μm) skin during period τ

| τ | X SKIN |
|---|---|
| 10 ns | 0.03 |
| 100 μs | 3.23 |
| 100 ms | 102 |

The thermal diffusivities (cm²s⁻¹) used in the calculation for skin is 0.001.

Neglecting the energy loss due to dissipation, the instantaneous temperature rise in a light absorbing medium at the end of a 10 ns laser illumination can be expressed as

ΔT=ααφ/ρc (2)

where αₐ in cm⁻¹ is the absorption coefficient; and φ in J/cm² is the laser fluence.

Penetration of radiation into a scattering medium(skin can be considered as a good example) is defined by the absorption of the radiation in this medium and by its scattering properties. The penetration depth d is expressed by the following formula:

d=1/(3αₐ(αₐ+αₛ′))^{1/2} (3)

where and αₐ is an absorption coefficient and αₛ is scattering extinction.

Geometrical dimension of beam size D of a thermal cavity preferably should be larger or equal penetration depth defined by (3). The equation (1) defines critical temporal relationship between dimensions of a thermal cavity and pulse duration of radiation that forms it.

According to the equation (1) pulse duration t is to satisfy the relationship:

D>d>\sqrt{4χτ} or τ<d²/4χ<D²/4χ

Droplets of electromagnetic energy are to satisfy the following relationship:

Δ<D, where Δ penetration depth of droplets which is in turn depends on the wavelength of the radiation, δ<d, δ is a diameter of the droplets. According to this relationship pulse duration of droplets should preferably be shorter compare to pulse duration of the pulse forming the thermal cavity. Thus, the thermal cavity and energy droplets can be formed by two (or more) types of radiation with different absorption in the medium or by the same radiation with different pulse duration or by a combination of both or by beam configuration. Then, following the equation (2) and definition of the thermal cavity, energy density and/or temperature in the droplets of energy is greater compare to the energy density and/or temperature in the thermal cavity giving a sense of using term ‘droplets’ to describe such a specific distribution of energy in tissue.

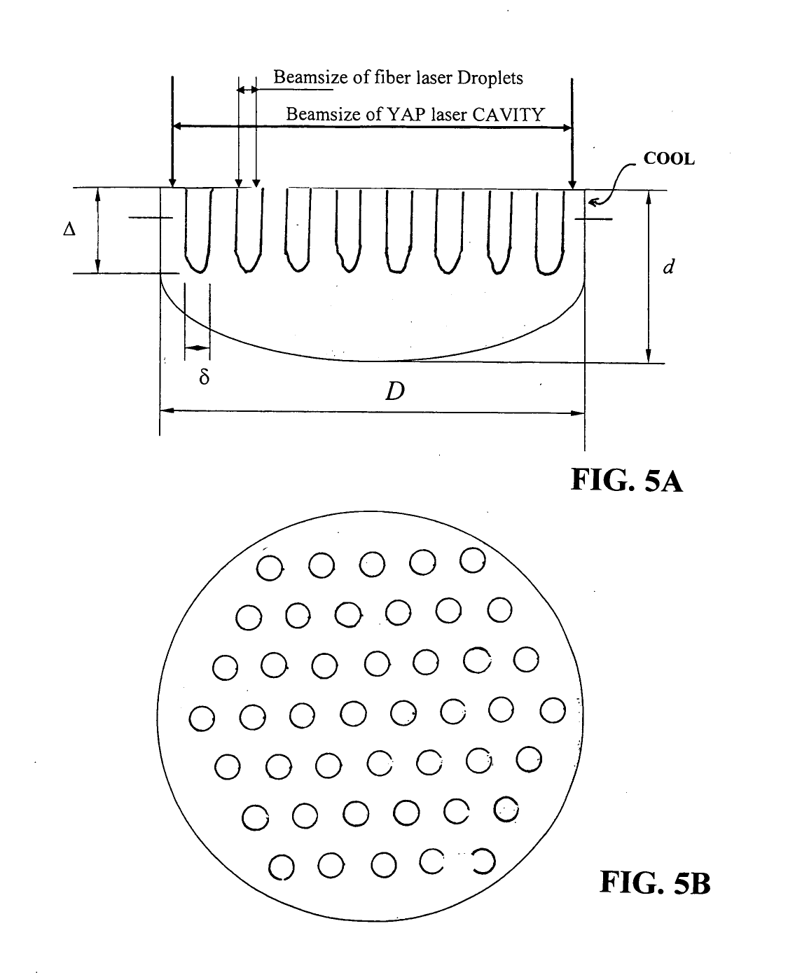

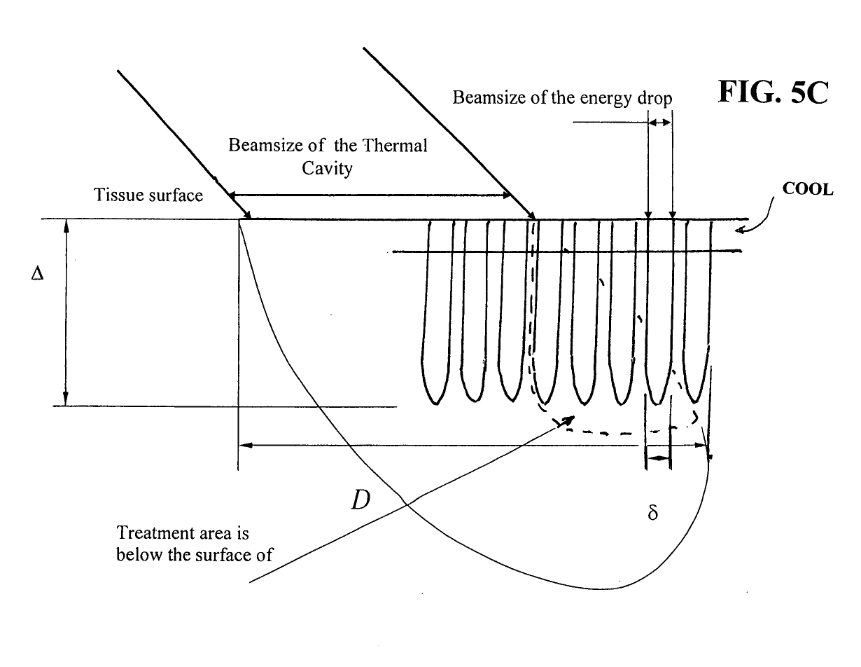

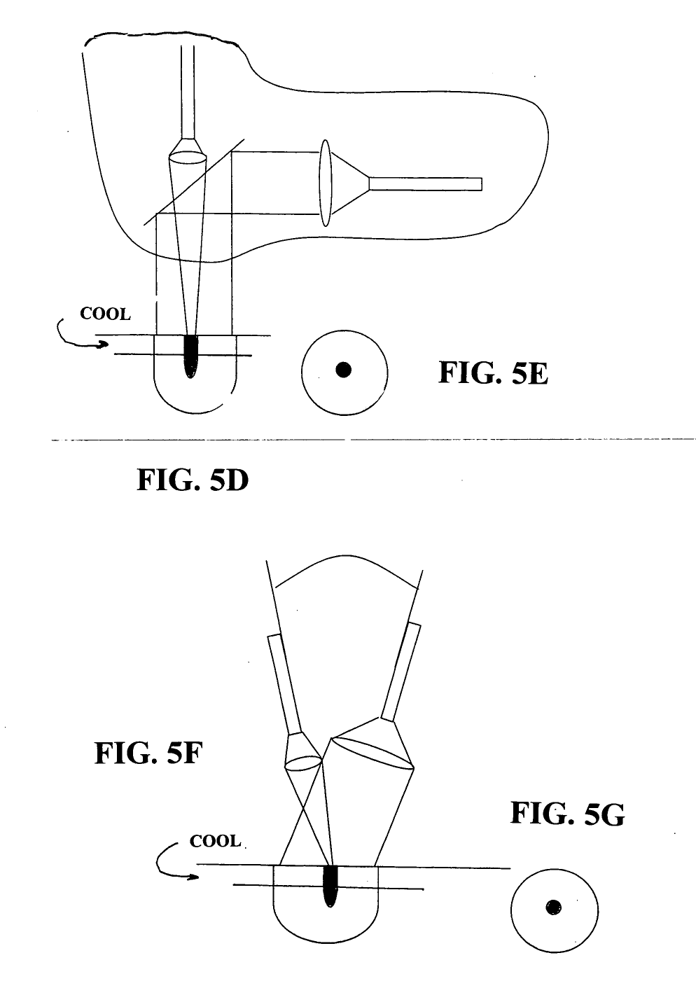

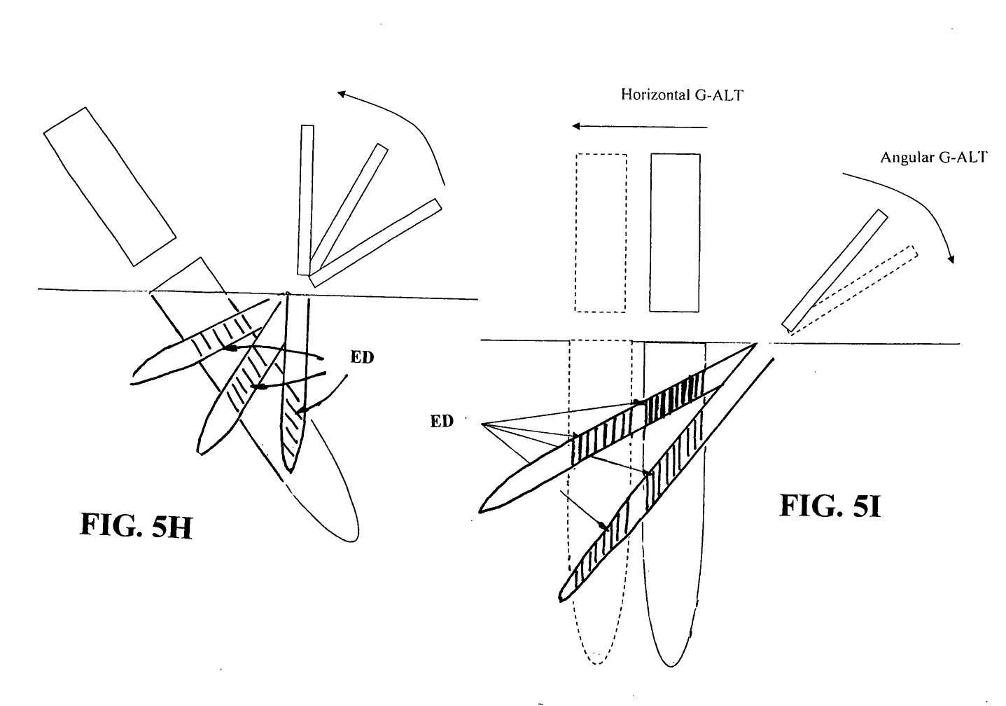

Such specific distribution of energy is essential to a number of aesthetic applications of lasers and other sources of radiation. Radiation that forms the thermal cavity does not damage the tissue, but increase overall energy deposition close to the damage threshold. In this case energy droplets provide more accurate energy delivery which is just needed for specific therapeutic effects due to supra threshold effects and spatial modulation of those effects across thermal cavity area. In specific embodiments energy droplets can be formed fixed as multi-beams crossing in the thermal cavity as shown at the FIG. 1A or they can be form via other techniques such as those indicated in FIGS. 5A through 5I.

### Hand Piece

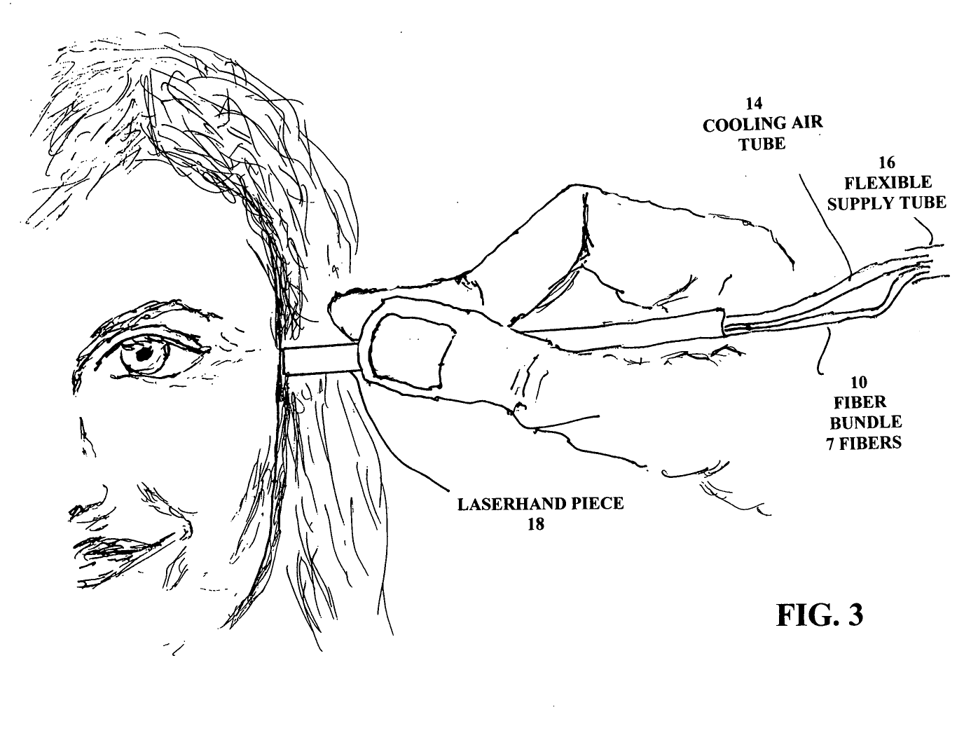

FIG. 3 shows the system in operation with the hand piece being used to treat the face of a patient. This embodiment damages tiny volumes of skin tissue about 0.5 mm below the surface of the skin. There is no significant damage to the skin surface or to any other portions of the skin outside the energy droplet. The damaged skin tissue will cause an immune response in a form of release of cytokines and stem cell factors which will create stem cell action producing new skin tissue resulting in younger looking skin. Since the skin is not damaged at the surface scaring or other surface effects are extremely unlikely.

### Laser System from Parent Application

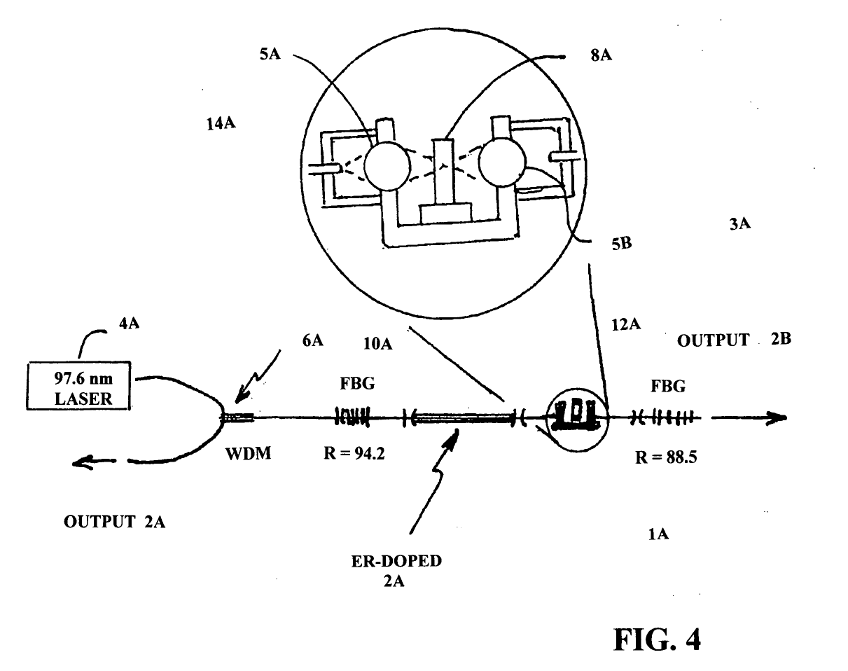

FIG. 4 is an optical schematic of Q-switched erbium laser described in detail in the parent patent application which is useful in an embodiment of the present invention. A laser cavity 1A is created with single mode erbium doped optical fiber 2A with a core diameter of 6.0 μm and a fiber length of anywhere between 0.2 m and 20 m. Applicant's preferred length is about 1 meter. The doping concentration of erbium ions is sufficient to produce about 1 dB/m to 350 dB/m absorption of the pump wavelength. Radiation from a pump diode laser 4A at a wavelength of 976 nm is launched with a wave divider multiplexer (WDM) coupler 6A into the master cavity. A Co²⁺:ZnSe crystal 8A with initial transmittance Tᵢₙ=70-98% and a thickness 0.3 mm-1 mm is positioned within the cavity and the cavity is defined by two fiber Bragg grating mirrors 10A and 12A with maximum reflection of 94.2% (100-95%) and 88.5% (70-98%), respectively, both gratings are designed for a wavelength of 1560 nm. The laser includes a U-bench unit 14A with a Co²⁺:ZnSe crystal inside as shown in FIG. 4.

U-bench 14A is a holder having a “U” shape as shown in FIG. 1, which is placed in between two ends of fiber tips and holds those fiber ends. The fiber ends are polished at a small angle of about 9 degrees to the optical axis to exclude back reflection. A small ball type lens 5A with a focal length of about 2 mm is placed at about a double focus distance from the fiber tip as shown in FIG. 4. The lens relays the image of the fiber tip to the center of U-bench. Then the image of the first fiber tip is relayed back onto the tip of the fiber positioned on the opposite side of U-bench 14A by a second ball type lens 5B. The beam waist is about 15 μm. All surfaces of optical elements including the Co²⁺:ZnSe crystal are coated to decrease reflection in the range of 1400-1800 nm. The system is aligned during fabrication. The inserted loss by introduction of a U-bench in to the fiber could be as low as 0.1 dB. In this preferred embodiment, the power density is adjusted so that the intra-cavity radiation level at the center of U-bench (i.e., the center of the Co²⁺:ZnSe) is about 1 kW/cm². Other parameters of Co²⁺:ZnSe crystal are:
1) absorption cross-section σₛ=5.3×10⁻¹⁹ cm²;
2) upper level lifetime τₛ=0.29×10⁻³ s;
3) bleaching power for Co²⁺:ZnSe, as low as 0.8 kW/cm².

The dependences of pulse energy (and, consequently, average power) of the laser can be estimated using the following formula for output energy of the laser operating in the passive Q switch mode:

$${E}_{out} = - \frac{{hvS}_{a}}{4 {σ}_{a} γ} ln \frac{1}{{R}_{1}} ln \frac{{n}_{a}^{min}}{{n}_{a}^{max}} ,$$

where Eₒᵤₜ is addressed to one of the laser outputs closed by the mirror M₁; nₐ^{min} and nₐ^{max} are the extremum inversion populations in AM; and h v is the energy of a laser output photon. Additional factor “2” in the denominator is introduced for accounting the Gaussian distribution of the beam in the laser cavity. The pulse energy of the pump laser is approximately constant so that pump energy is roughly proportional to pump rate. A minimum of giant pulse duration and maxima of the pulses' energy and peak power are observed close to the middle point of the passive Q switch mode. This fact allows one to manipulate with the output parameters of the laser by simply changing the pump rate.

The laser threshold was measured to be 19.3 mW at wavelength 1559.5 nm, where the laser operates in the super-luminescence regime. Just above the threshold of oscillation, with pump power increased up to 20.5 mW, the laser transited to the passive Q switched regime, where stable giant pulses are generated. Rather long pulse width of the giant pulses is the result of a considerably long length of the cavity. Thus, the pulse duration can be controlled to an extent by choosing the length of the cavity. Or the laser may be designed to provide maximum total pumping of the fiber to produce maximum pulse power. Pulse duration could also be shortened using a high-doped erbium fiber of short length (less than 2 m) as an active medium of the laser. In this case pulse duration could be in the range 0.5 μs-3 μs. The repetition rate of the pulses in a train increases with the pump repetition rate up to about 50 kHz.

A Cr²⁺:ZnSe crystal for Q-switch could be substituted for Co²⁺:Zn. The parameters of the U-bench were chosen to produce power density of the intra-cavity radiation in the center of U-bench at about 60 kW/cm². The crystal Cr²⁺:ZnSe was placed near the center of U-bench to provide location of the beam waist of 1 μm close to the crystal center. The Cr²⁺:ZnSe crystal had antireflection coating at wavelength 1400-1800 nm. A sample of Cr²⁺:ZnSe crystal with initial transmittance Tᵢₙ=50-98% and thickness 0.3-1 mm, and the two fiber Bragg grating (FBG) mirrors with maximum of reflection of 100-95% and 70-98%, respectively. The bleaching power of a Cr²⁺:ZnSe is 60 kW/cm². Using this passive Q-switch modulator pulse duration as short as 10 ns to 500 ns might be obtained depending on the pump rate and the length of the fiber laser.

Thus, by changing pump rate, concentration of Er dopping in the fiber, length of laser resonator(fiber) and a type of passive Q-switcher it is possible to vary pulse duration of the fiber laser in the range from 10 ns to 15 μs.

### Other Alternatives

A preferred wavelength of YAP:Nd laser is 1079 nm, energy fluence 20 J/cm² and pulse duration 1 ms. In this preferred embodiment individual fiber laser operates at the output energy level 300 mW individual pulse duration 2 μs, burst duration 900 μs. Both YAP:Nd and fiber laser are synchronized to deliver laser pulses approximately at the same time. Mismatch of synchronization or delay between pulses should be less than thermal relaxation time defined in formula (1), and in this case this time equals to 0.5 ms. The Perovskite laser for this preferred embodiment is available at Fotona Lasers, Ljubljana, Slovenia and the fiber laser is available from IPG Photonics at Waltham, Mass.

Battery operated hand held systems are available for producing thermal cavity with energy droplets for skin rejuvenation. In those systems energy is provided by powerful laser diodes and they are available from Intenzity with office in Vancouver, BC, Canada. In this preferred embodiments laser diodes at about 980 nm maximum emission band is used to produce thermal cavity and several diodes (e.g. 1 to 4) with maximum emission around 1290 nm are used to produce energy drops. Those laser diodes at 980 and 1290 nm could be combined in one pack. The electrical power for those diodes is provided by batteries with 3V voltage rating and a control circuit. The power current needed for diode operation is about 2-3 A. This level of current is provided by current alkaline or Li-ion(lithium) type AA batteries and even re-chargeable NiMeH (nickel metal hydrate)batteries. Thus, a combination of laser diodes, batteries, control circuit and lens focusing system forms a compact hand-held system.

Powerful laser diodes in wavelength range 1290 nm are available from Covega Corporation Jessup, Md., powerful laser diodes at 980 nm are available from Spectra Physics, Mountain View, Calif.; Coherent, Santa Clara, Calif. Alkaline or re-chargeable NiMeH batteries are available from Energizer or other off-the-shelf suppliers.

### Other Optics Units

In preferred embodiments each system is provided with a set of optics units 20 each providing for the energy droplet at a different location below the skin surface. Preferably, the locations will cover a range below the surface between about 0.2 mm to about 1.0 mm. The number of laser beams carrying the energy of the second laser source could be as few as two to as many as ten. The energy of the first laser source could also be divided into a number of separate fibers.

### Other Beam Patterns

Many other crossing beam patterns are possible using the teachings of this invention to form energy droplets under the skin. Some of these are suggested in FIGS. 5A through 5I. FIGS. 5A and 5B show a pattern created by 44 short pulse narrow beams directed into a large energy cavity. Preferably the surface of the skin is cooled so that 44 energy droplets are created in tiny regions about 1 to 3 millimeters below the skin surface.

FIG. 5C shows a technique similar to the one shown in 5A and B, but here the energy cavity is created by a laser beam directed at an angle to assure that most of the energy droplets are created below the skin surface.

FIGS. 5D and 5E demonstrate a technique for using a beam splitter to permit the cavity creating beam and the energy droplet beam to be co-aligned.

FIGS. 5F and 5G is a similar technique where the two beans are angularly offset.

FIGS. 5H and 5I show techniques where one thermal cavity laser beam and one energy droplet beam are adjusted to create multiple energy droplets below the skin surface.

### Other Applications of the Present Invention

The above embodiments describe techniques for skin treatments based on the concept that by utilizing thermal cavities and energy droplets to intentionally cause damage to tiny volumes of skin below the surface of the skin, natural skin renewal processes will be initiated in the skin that will extend beyond the region damaged to produce rejuvenated skin. This concept can be extended to other types of treatments. Some of these other types of treatments are discussed below:

### Large Area Skin Rejuvenation

For large area skin rejuvenation a combination of 980 nm and 1440 nm is recommended. The 980 nm beam is to form energy cavity with deep penetration (up to 5 mm) and the 1440 nm beam or beams is to form energy drops with penetrations of about 0.33 mm. This combination is for large area skin rejuvenation including neo-collagen formation, sebaceous gland and bulge area stem cell mobilization.

### Treatment of Pigmented Lesions and Cellulite Treatments

Applicants recommend a combination of 670 nm beam and 915 nm beam for treatment of pigmented lesions and cellulite treatments. The 915 nm beam is to form energy cavity with deeper penetration (up to 4 mm) and the 670 nm beam is to form energy droplets with penetration is less than 1 mm. The 915 nm beam penetrates deep into skin tissue and increases temperature and thus help to liquefy subcutaneous and the 670 nm beam increases cell membrane permeability and helps liquefied fat to move into the interstitial space. The surface of the skin should be cooled enough to prevent surface damage form the 670 nm beams.

### Treatment of Acne Scar Tissue

Applicants recommend 980 nm and 1930 nm for acne scar treatment. The 980 nm beam is to form the energy cavity with penetration several mm and the 1930 nm beam is to form energy drops with penetration is just 0.08 mm. As above the skin surface should be cooled to prevent surface damage. This combination can also be used for skin rejuvenation.

### Hair Removal

For a hair removal technique Applicants recommend a combination of 810 nm and 980 nm. The nm 980 nm beam is to form energy cavity and 810 nm beam is to form energy droplets. The 810 nm beam is for pigmented hair tissue, shaft and matrix, while the 980 nm beam is deeper penetration and less absorption in melanin for outside hair root channel tissue that contains blood vessels and stem cells. The 810 nm energy is strongly absorbed in the pigmented hair tissue and thus damages the hair tissue while at the same providing the thermal cavity. The purpose of the 980 energy droplets is to stimulate new skin tissue growth in the treated region of the skin. This combination is also good for skin rejuvenation.

### Treatment of Rhytides and Acne and Acne Scar Tissue

Applicants recommend a combination of 457 nm, 980 nm and 1440 nm for treatment of rhytides and acne and acne scar tissue. The 980 nm beam is to form energy cavity and the 1440 nm and 457 nm beams are to form multiple color energy drops.

### Treatment of Telangiatesia and Pigmented Lesions

Applicants recommend 980 nm and 532 nm or 540 nm for telangiatesia and pigmented lesions treatments. The 980 nm is to form energy cavity and 532 nm or 540 nm is to form energy drops. Wavelengths in the range of 532-540 nm are strongly absorbed by hemoglobin in the blood and helps coagulation of blood vessels and 980 nm provides uniform preheating of and around the vessels and blood plasma.

### Pigmented Lesions and Wrinkles

For treatment of pigmented lesions and wrinkles Applicants recommend a combination of 980 nm, 532 nm and 1440. The 980 nm beam is to form energy cavity with deep penetration (several mm) and 532 nm and/or 1440 nm are to form energy drops to treat pigmented lesions and wrinkles at the same time.

### Treatment of Ulcers

For treatment of ulcers Applicants recommend 890 nm and 640 nm. The 890 nm beam provides thermal cavity and the 640 nm energy is well absorbed in melanin and therefore pigmented hairs, 890 nm is to provide uniform heat distribution outside hair shaft. This combination could be used for hair removal where the 640 beam is directed at the hair root. This combination can also provide heat modulation for treatment of different type of ulcers (external and internal).

### Collagen Modification

For collagen modification Applicants that 1440 nm beams be used for both the thermal cavity and the energy droplets. For the thermal cavity use a wide beam and long pulse and for the energy droplets use multiple narrow beams and of short pulses. These combinations can also be used for skin rejuvenation. Application is enhanced collagen modification for skin rejuvenation.

### Treatment of Scar Tissue and Stretch Marks

For these types of treatment, Applicants recommend a wavelength combination of 1440 nm and 1930 nm. The 1440 nm beam is to form the energy cavity with penetration up to the junction of papillary and reticular dermis (0.33 mm) and the 1930 nm beam is used to form energy droplets. The absorption of the 1930 nm beam is 4 times stronger than the absorption of the 1440 nm beam (penetration 0.08 mm) for the opening epidermal layer and cell delivery.

### Laser Suppliers

All of the above combinations of wavelengths can be obtained by using powerful laser diodes. The laser diodes can be obtained from a number manufacturers like nLight, with offices in Vancouver, Canada; Coherent, with offices in Santa Clara, Calif. and LPG Photonics, with offices in Oxford, Mass.

Other laser suppliers include Palomar (Burlington, Mass.), Cynosure (Westford, Mass.), Candela (Wayland, Mass.), Eleme(Merrimack, N.H.), Sciton (Palo Alto, Calif.), Lumenis (Santa Clara, Calif.), Cutera (Brisbane, Calif.).

Table II below summarizes some preferred wavelength ranges, laser pulse energy and pulse rate ranges for both the first laser source to produce the thermal cavity and the second laser source to produce energy drop. The value of laser energy is that measured before the light is absorbed in skin tissue. The amount of energy absorbed in the skin is same for all lasers and such to produce a temperature rise about 5° C. for the thermal cavity with the diameter of the thermal cavity D is 10 mm. The diameter of the energy drop d is 2 mm and average temperature rise is 10° C. If the energy E is too high to obtain from a laser system the spot size should be decreased. Laser Energy is proportional to laser spot size diameter squared (E˜D², E˜d²).

TABLE II

| Preheat Laser Source | | | Energy Droplet Laser source | | | |
|---|---|---|---|---|---|---|
| | Laser Energy, | Laser Pulse | | Laser Energy, | Laser Pulse | |
| Wavelength | Joules | duration range | Wavelength | millijoules | duration range | |
| 890 nm | 27 | 1-20 ms | 640 nm | 330 | 0.01-1 | ms |
| 915 nm | 33 | 1-20 ms | 670 nm | 440 | 0.01-1 | ms |
| 980 nm | 41 | 1-20 ms | 532-540 nm | 110 | 0.01 mcs-1 | ms |
| 980 nm | 41 | 1-20 ms | 457 and 1440 nm | 44 and 66 | 0.01-1 | ms |
| 980 nm | 41 | 1-20 ms | 1930 nm | 17 | 0.1 mcs-1 | ms |
| 980 nm | 41 | 1-20 ms | 1290 nm | 880 | 0.1 mcs-1 | ms |
| 1064 nm | 54 | 0.1-50 ms | 532 nm | 110 | 0.01 mcs-1 | ms |
| 1064 nm | 54 | 0.1-50 ms | 1320 nm | 660 | 0.1 mcs-1 | ms |
| 1064 nm | 54 | 0.1-50 ms | 1440 nm | 66 | 0.1 mcs-1 | ms |
| 1079 nm | 82 | 0.1-50 ms | 1540 nm | 102 | 0.01 mcs-1 | ms |
| 1440 nm | 0.82 | 1-20 ms | 1930 nm | 17 | 0.1 mcs-1 | ms |
| 1440 nm | 0.82 | 1-20 ms | 1440 nm | 66 | 0.1 mcs-1 | ms |

The reader should understand that the above specific embodiments of the present invention are merely examples and that many changes and modifications could be made without departing from the important concepts of the present invention. For example, many sources of radiation at different wavelengths that are scattered and absorbed in tissue and skin with specific configuration and time relationship may be substituted for the lasers described in detail. Preferred ranges for the preheat laser beam is a laser beam with a peak wavelength chosen from the following group of wavelengths: 750 nm to 1450 nm, 1500 nm to 1850 nm, 2200 nm to 2300 nm; and preferred ranges for the energy droplet laser beam is a laser beam with a peak wavelength chosen from the following group of wavelengths: 200 nm to 750 nm, 1400 nm to 1600 nm, 1850 nm to 3000 nm. In fact the above-described source of radiation could be any source of electromagnetic energy that meets the above-described criteria, such as microwave, radio frequency, light (laser diodes, light emitting diodes (LEDs), non-coherent light source), etc. Those sources of radiation could be combined to produce at least two wavelengths to form thermal cavity and energy drops. The laser hand piece could be pre-cooled to close to freezing and pressed against the skin prior to illumination to provide skin surface cooling. Therefore, the reader should determine to scope of the present invention by the appended claims and their legal equivalents.

We claim:

1. A laser tissue treatment process, comprising the steps of:
A) illuminating a first region of skin, defining a skin surface, for a first time period with at least one preheat laser beam with sufficient energy to heat the first region to a first temperature-time profile that is close to but below a temperature-time profile that would cause tissue damage, said first region so heated with said first temperature-time profile defining a thermal cavity; and
B) illuminating at least one sub-region of skin located below the skin surface and within said first region of skin and for a second time period with at least one energy droplet laser beam adapted to apply additional quantity of heat to said at least one sub-region of skin to increase the temperature-time profile in said at least one sub-region to a temperature-time profile in excess of a temperature-time profile sufficient to cause tissue damage within said at least one sub-region, each of said additional quantity of heat in each of said at least one sub-region defining an energy droplet;
wherein said tissue damage in said at least one sub-region stimulates a healing process that subsequently extends beyond the region damaged to produce rejuvenated skin tissue.

2. The laser tissue treatment process as in claim 1 wherein said at least one energy droplet laser beam is a plurality of laser beams.

3. The laser tissue treatment process as in claim 2 wherein said plurality of energy droplet laser beams is six energy droplet laser beams.

4. The laser tissue treatment process as in claim 1 and further including a step of cooling the skin surface.

5. The laser tissue treatment process as in claim 1 and further including a step of cooling the skin surface prior to the steps of illuminating the first region of skin.

6. The laser tissue treatment process as in claim 5 wherein said preheat laser beam is a laser beam with a peak wavelength chosen from the following group of wavelengths: 750 nm to 1450 nm, 1500 nm to 1850 nm, 2200 nm to 2300 nm; and said energy droplet laser beam is a laser beam with a peak wavelength chosen from the following group of wavelengths: 200 nm to 750 nm, 1400 nm to 1600 nm, 1850 nm to 3000 nm.

7. The laser tissue treatment process as in claim 5 wherein said at least one preheat laser beam and said at least one energy droplet laser beam in terms wavelength, laser energy and pulse duration are provided respectively by laser sources listed in the following table:

| Preheat Laser Source | | | Energy Droplet Laser source | | | |
|---|---|---|---|---|---|---|
| | Laser Energy, | Laser Pulse | | Laser Energy, | Laser Pulse | |
| Wavelength | Joules | duration range | Wavelength | millijoules | duration range | |
| 890 nm | 27 | 1-20 ms | 640 nm | 330 | 0.01-1 | ms |
| 915 nm | 33 | 1-20 ms | 670 nm | 440 | 0.01-1 | ms |
| 980 nm | 41 | 1-20 ms | 532-540 nm | 110 | 0.01 mcs-1 | ms |
| 980 nm | 41 | 1-20 ms | 457 and 1440 nm | 44 and 66 | 0.01-1 | ms |
| 980 nm | 41 | 1-20 ms | 1930 nm | 17 | 0.1 mcs-1 | ms |
| 980 nm | 41 | 1-20 ms | 1290 nm | 880 | 0.1 mcs-1 | ms |
| 1064 nm | 54 | 0.1-50 ms | 532 nm | 110 | 0.01 mcs-1 | ms |
| 1064 nm | 54 | 0.1-50 ms | 1320 nm | 660 | 0.1 mcs-1 | ms |
| 1064 nm | 54 | 0.1-50 ms | 1440 nm | 66 | 0.1 mcs-1 | ms |
| 1079 nm | 82 | 0.1-50 ms | 1540 nm | 102 | 0.01 mcs-1 | ms |
| 1440 nm | 0.82 | 1-20 ms | 1930 nm | 17 | 0.1 mcs-1 | ms |
| 1440 nm | 0.82 | 1-20 ms | 1440 nm | 66 | 0.1 mcs-1 | ms |

8. The laser tissue treatment process as in claim 1 wherein the energy droplet laser beam is a laser beam having a peak wavelength of about 1079 nm and the preheat laser beam is a laser beam having a peak wavelength of about 1560 nm.

9. The laser tissue treatment process as in claim 7 wherein said at least one energy droplet laser beam is a plurality of energy droplet laser beams.

10. The laser tissue treatment process as in claim 9 wherein said plurality of energy droplet laser beams is six energy droplet laser beams.

11. The laser tissue treatment process as in claim 4 where said skin surface is cooled with cold air.

12. The laser tissue treatment process as in claim 5 wherein said at least one preheat laser beam and said at least one energy droplet laser beam are applied via a laser hand piece.

13. The laser tissue treatment process as in claim 12 wherein said laser hand piece is pre-cooled and said skin surface is cooled through physical contact with the laser hand piece.

14. The laser tissue treatment process as in claim 12 wherein at least one preheat laser beam and said at least one energy droplet laser beam are delivered via optical fibers.

15. The laser tissue treatment process as in claim 1 wherein said process is adapted to treat a condition chosen from the following group of skin conditions:
A) pigmented lesions
B) cellulite
C) acne scaring
D) other scaring
E) unwanted hair
F) rhytides
G) telangiatesia
H) wrinkles
I) skin ulcers
